(19) United States
(12) Patent Application Publication
(10) Pub. No.: US 2024/0066089 A1
(43) Pub. Date: Feb. 29, 2024

(54) ARTOCARPUS HETEROPHYLLUS COMPOSITION AND METHOD OF PROMOTING URINARY TRACT HEALTH

(71) Applicant: Kodimule Shyam PRASAD, Bangalore (IN)

(72) Inventor: Kodimule Shyam PRASAD, Bangalore (IN)

(21) Appl. No.: 18/215,826

(22) Filed: Jun. 29, 2023

Related U.S. Application Data
(60) Provisional application No. 63/391,928, filed on Jul. 25, 2022.

(51) Int. Cl. A61K 36/60; A61K 31/353; A61K 31/56; A61K 31/575; A61P 13/08

(52) U.S. Cl. CPC A61K 36/60; A61K 31/353; A61K 31/56; A61K 31/575; A61P 13/08

(57) ABSTRACT

The invention provides a composition comprising an extract of Artocarpus heterophyllus and methods using and manufacturing the composition in the treatment of lower urinary tract symptoms (LUTS). The extract can be derived from the leaves of Artocarpus heterophyllus and can be formulated with carriers, excipients, nutritional supplements, food supplements, and beverages. The extract finds use in the treatment of LUTS associated with prostate disorders, such as benign prostatic hyperplasia. The composition can similarly be administered in methods of promoting and maintaining the health of the urinary tract and prostate.

### CROSS-REFERENCE TO RELATED APPLICATIONS

This application claims the benefit of provisional application No. 63/391,928 filed Jul. 25, 2022, the entire contents of which are incorporated herein for all purposes.

### FIELD OF INVENTION

The present invention relates to a plant-based composition and method for its use and manufacture. More particularly, the invention relates to a composition comprising an extract of Artocarpus heterophyllus and method for its use and manufacture in the treatment of lower urinary tract symptoms (LUTS).

### BACKGROUND OF THE INVENTION

LUTS is a group of urinary symptoms triggered by an obstruction, abnormality, infection or irritation of the urethra, bladder, bladder neck, urinary sphincter and/or prostate in men. LUTS can also be caused by a neurological health condition or injury. LUTS can be categorized as being related to urine storage or voiding. LUTS can present themselves as various voiding dysfunctions. Both men and women are diagnosed with LUTS, but it is most often diagnosed in men with benign prostatic hyperplasia (BPH). LUTS is not a normal part of the aging process, but it is diagnosed more often as people age.

BPH is a deteriorating health condition characterised by benign enlargement of prostate in older men (Roehrborn, 2011; Wu and Kapoor, 2013). BPH is characterized by the increased proliferation of stromal and glandular epithelial cells leading to the enlargement of prostate size and weight (Schauer and Rowley, 2011). BPH is mediated by the conversion of circulating testosterone to dihydrotestosterone (DHT) by 5a-reductase in the prostate via regulation of androgen receptor transcription (Chughtai et al. 2016).

Phytotherapy could be of extreme help in combating LUTS due to its multi-targeted nature. Medicinal plants such as Serenoa repens, Urtica dioica and Pygeum africanum have been documented for treatment of BPH symptoms. Other promising herbs include Epilobium spp. (Vitalone and Alkanjari, 2018), Cucurbita pepo, Vaccinium macrocarpon, and Lycopersum esculentum.

Artocarpus heterophyllus Lam., (Fam. Moraceae) is a tropical tree species known for its edible fruit called Jackfruit. Artocarpus heterophyllus (A. heterophyllus) is distributed in Asia, Africa and some parts of South America (Baliga et al. 2011). It is widely consumed in Western Ghats of India (Jagadeesh et al. 2007). A. heterophyllus contains several groups of phytochemicals such as carotenoids (Chandrika et al. 2004), flavonoids (Venkataraman, 2001), sterols, volatile acids and tannins (Wong et al. 1992). Jackfruit has several high-value compounds with physiological functions (Jagtap et al. 2010). It is reported to have antimicrobial, anti-inflammatory, anti-diabetic, and antioxidant activities (Shanmugapriya et al. 2011). Jackfruit is a rich source of flavonoids attributing to its potent anti-inflammatory activity.

Parts of A. heterophyllus including fruits, leaves and stem bark have been used in traditional medicine. The present invention describes the novel application of the plant in the treatment of LUTS, including LUTS resulting from BPH.

### SUMMARY OF THE INVENTION

The inventor surprisingly discovered a novel composition derived from A heterophyllus and method of its use and manufacture in the treatment of LUTS, including BPH.

It is therefore an object of the invention to provide a composition comprising an extract of A heterophyllus for the treatment of LUTS.

A further object of the invention is to provide a method of treating LUTS, comprising administering to a subject in need thereof an effective amount of a composition comprising an extract of A heterophyllus.

In some aspects, the subject is at risk of developing LUTS and administering the composition inhibits, prevents or delays the symptoms of LUTS.

In some aspects of the invention, the LUTS is the result of BPH.

In some aspects of the invention, administering the composition prevents or inhibits increases in prostate size, decreases prostate size, prevents or inhibits increases in prostate index, reduces prostate index, prevents or inhibits increases in serum testosterone levels, reduces serum testosterone levels, prevents or inhibits BPH prostate pathology, or reverses BPH prostate pathology.

In another aspect, the composition can be used to inhibit 5α-reductase activity, particularly type 2 5α-reductase activity.

In yet another aspect, the invention provides a method of promoting and maintaining urinary tract health, comprising administering a composition comprising an extract of A heterophyllus as disclosed herein.

### BRIEF DESCRIPTION OF DRAWINGS

FIG. 1 shows an HPLC chromatogram of β-sitosterol in an embodiment of the inventive composition.

FIG. 2 shows an LCMS/MS fingerprint chromatogram of phytosterols in an embodiment of the inventive composition.

FIG. 3 shows an LCMS/MS fingerprint chromatogram of flavonoids in an embodiment of the inventive composition.

FIGS. 4A and 4B show the inhibitory effect of different concentrations of an embodiment of the inventive composition versus reference drug Finasteride against 5α-reductase activity in vitro.

FIGS. 5A, 5B and 5C shows the effect of the administration of an embodiment of the inventive composition on body weight, prostate weight, and prostate index respectively in testosterone-induced BPH model rats.

FIG. 6 shows the serum testosterone levels in BPH model rats treated with an embodiment of the inventive composition.

FIG. 7 shows the effect of the administration of an embodiment of the inventive composition on 5α-reductase activity in the prostate tissues of testosterone-induced BPH model rats.

FIG. 8 shows the histomorphology of prostate tissues of BPH model rats in different treatment groups.

FIG. 9 shows the effect of the administration of an embodiment of the inventive composition on the antioxidant status in prostate tissues of testosterone-induced BPH model rats.

FIG. 10 shows the chemical structures of major flavonoids present in an embodiment of the inventive composition.

FIG. 11 shows the crystal structure of human liver 5-β-reductase enzyme.

FIG. 12 shows the interactive pattern of flavonoid components of an embodiment of the inventive composition versus Finasteride with active site residues of 5-β-reductase.

### DETAILED DESCRIPTION

The present invention relates to the inventor's surprising discovery that a composition comprising A heterophyllus extract is effective in the treatment of LUTS and BPH. Without being limited to any particular theory or mechanism, flavonoids contained within A heterophyllus extract impart a therapeutic effect by inhibiting the activity of 5-alpha-reductase. The inventor surprisingly discovered, and has exemplified herein, that the flavonoids artocapanone, artocarpin and cycloartocarpin can interact with and block with the 5-alpha-reductase active site. The results obtained by the inventor unexpectedly showed that the flavonoids of the composition can be comparable to Finasteride in their affinity for the 5-alpha-reductase active site. Without being limited to any particular theory or mechanism, administering the inventive composition inhibits the activity of 5-alpha-reductase and modulates serum testosterone levels thereby preventing or inhibiting prostate growth and accompanying LUTS.

The extract for use with the inventive composition can be obtained from any part of the A heterophyllus plant. The extract can be obtained from one or more of the plant's fruit, fruit skin, seeds, leaves, bark, wood, stems, roots and flowers. In a preferred embodiment, the extract is obtained from A heterophyllus leaves. The plant parts can be fresh, dried, or partially dried. The plant parts can be whole, crushed, chopped, or in the form of a powder.

The extract can be obtained by solvent extraction of one or more plant parts of A heterophyllus. Extraction can be performed with one or more aqueous solvents, one or more alcohol-based solvents, one or more organic solvents, one or more supercritical fluids, or combinations thereof. Non-limiting examples of alcohol-based solvents for use with the invention include, but are not limited to, ethanol and methanol. Alcohol-based solvents for use with the invention can be hydrous or anhydrous. Supercritical fluids for use with the invention include, but are not necessarily limited to, carbon dioxide.

In some aspects, the extract comprises one or more phytosterols, one or more flavonoids, or a combination thereof. The phytosterols can be one or more of (3-sitosterol, ergosterol, lupeol, campestenol, campesterol, and stigmasterol. The flavonoids can be one or more of artocapanone, artocarpin and cycloartocarpin. The extract can comprise at least about 3.0% w/w β-sitosterol, or at least about 5.0% w/w β-sitosterol. In some aspects, the extract comprises about 3.5±0.5% w/w β-sitosterol, or between about 3.0% and 5.0% w/w β-sitosterol.

In some aspects of the invention, the composition comprises an extract of A heterophyllus and at least one excipient. The excipient can be selected on the basis of compatibility with the extract and the properties of the desired dosage form. Suitable excipients include, but are not necessarily limited to, carriers, binders, fillers, flow aids/glidents, disintegrants, lubricants, stabilizers, surfactants, preservatives, diluents, and the like. Excipients for use with the invention include artificial excipients. Suitable excipients include, but are not necessarily limited to, those disclosed in the following references: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). The entire contents of these references are incorporated herein by reference for all purposes. The composition can include a sweetener, flavor, vitamin, mineral, sugar, protein, amino acid, starch, or combinations thereof

In some embodiments, the composition can employ controlled, sustained, or extended release formulations known collectively as “modified release” formulations. Modified release formulations for use with the invention include, but not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the entire contents of which are incorporated herein by reference for all purposes. Administration forms for the composition can be used to provide modified release of one or more active ingredients using, for example, hydropropylmethyl cellulose, other polymer matrices, gels, permeable membranes, osmotic systems, multilayer coatings, microparticles, liposomes, microspheres, or a combination thereof

The composition can be in an administration form selected form a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, injection, or combinations thereof. The composition can be formulated for oral administration.

In some aspects of the invention, the composition is combined with a food, snack, nutritional supplement, dietary supplement, food supplement, or beverage. The food, snack, nutritional supplement, dietary supplement, food supplement, or beverage can be used by consumers as a means for promoting or maintaining urinary tract health or prostate health. The composition can be combined with snacks such as bars, chips, chews, gels, gums, candies, chocolates, cakes, cookies and other pastries, wafers, crackers, ice cream, and the like.

The composition can be provided in bulk quantities for the industrial manufacture of the products and administration forms described herein. For example, the composition can be provided in bulk quantities as a powder, cake, or liquid. Bulk quantities of the composition can be packaged, stored and/or distributed in drums, bags, boxes, and other containers which are adapted to preserve or otherwise prevent or inhibit the oxidation of one or more active ingredients of the composition.

The composition can assume one or more of the administration forms disclosed herein, wherein the composition is contained in a container. The container can include instructions for using the composition according to the methods disclosed herein. The instructions can be printed matter that is placed in and/or on the container.

In some embodiments, the invention provides a method of treating LUTS. The invention can be practiced by administering to a subject in need thereof an effective amount of a composition as disclosed herein. The subject can have, or be at risk of developing, one or more LUTS. Administering the composition can prevent the development of, delay the onset of, inhibit the progression of, or reverse one or more LUTS in the subject. LUTS treatable by the method of the invention include, but are not necessarily limited to, urgent need to urinate, increased frequency of urination, difficulty starting urination, weak urine stream, urine stream that stops and starts, dribbling at the end of urination, involuntary urination, painful urination, inability to completely empty the bladder, urinary tract infection, inability to urinate, blood in the urine, or combinations thereof. The subject treated for LUTS can have or be at risk of having a prostate disorder. The prostate disorder can be BPH, prostatitis, or a combination thereof. The subject can have one or more of an enlarged prostate, increased prostate weight, prostate inflammation, inhibited apoptosis of prostate tissue, and prostate growth.

In other embodiments, the invention provides a method of treating BPH, comprising administering to a subject in need thereof an effective amount of a composition as disclosed herein. The subject can have BPH, wherein administering the composition inhibits the progression of BPH or reverses BPH-associated conditions in the prostate of the subject. In some aspects, the subject can be at risk of developing BPH, wherein administering the composition prevents or delays the development of BPH in the subject. In some aspects, administering the composition in the treatment of BPH prevents, inhibits, or delay increases in serum testosterone levels and/or prostate growth.

In one non-limiting embodiment, the invention provides a method of promoting (e.g., maintaining) prostate health and/or urinary tract health in a subject in need or desirous thereof. Such method can be practiced by administering to the subject an effective amount of a composition disclosed herein. The subject can be a male subject. Administering the composition for maintaining prostate and/or urinary tract health can promote or maintain proper urinary tract function. Administering the composition for maintaining prostate and/or urinary tract health can prevent or delay the onset of one or more LUTS.

The composition can be administered to the subject such that the subject receives a dose of A heterophyllus extract that is between about 50 mg/kg b.w and about 60 mg/kg b.w., or between about 200 mg/kg b.w and about 400 mg/kg b.w. The composition can be administered such that the subject receives a dose of A heterophyllus extract that is between about 5 mg/day and about 500 mg/day. The composition can be administered such that the subject receives a dose of A heterophyllus extract that is about 5 mg/day, about 10 mg/day, about 15 mg/day, about 20 mg/day, about 21 mg/day, about 22 mg/day, about 23 mg/day, about 24 mg/day, about 25 mg/day, about 26 mg/day, about 27 mg/day, about 27 mg/day, about 28 mg/day, about 29 mg/day, about 30 mg/day, about 31 mg/day, about 32 mg/day, about 33 mg/day, about 34 mg/day, about 35 mg/day, about 40 mg/day, about 45 mg/day, about 50 mg/day, about 100mg/day, about 150 mg/day, about 200 mg/day, about 250 mg/day, about 300 mg/day, about 350 mg/day, about 400 mg/day, about 450 mg/day, or about 500 mg/day, as well as any dosage intervening these specifically disclosed amounts. The composition can be administered to the subject topically or systemically. The composition can be administered orally, buccally, sub-lingually, parenterally, intravenously, intranasally, intravaginally, rectally, by inhalation, or combinations thereof. In a preferred embodiment, the composition is administered orally.

Aspects of the invention are demonstrated through the following examples. It will be understood that these examples are not intended to be limiting in any way. One skilled in the art will appreciate that variations in the examples and embodiments disclosed in the detailed description are possible without departing from the invention encompassed by the appended claims.

Example 1 shows a process for preparing an embodiment of the extract for use with invention. Example 2 shows the characterization of phytochemicals in the extract prepared according to Example 1. Example 3 demonstrates the in vitro evaluation of 5-alpha-reductase inhibition by the extract prepared according to Example 1. Example 4 shows the efficacy of the extract prepared according to Example 1 in BPH model rats. Example 5 shows a molecular docking analysis of 5-alpha-reductase with flavonoids that are present an embodiment of the inventive extract.

### EXAMPLE 1

### Making Extract from A. heterophyllus Leaves

1000 kg of Artocarpus heterophyllus (jack fruit) leaves were pulverized to 3-5 mm powder in a hammer mill and stacked in a vertical 8.0 KL extractor. 5-6 KL of hydro alcohol was added and the extraction mass was refluxed at 65-70° C. about 7-8 h with continuous circulation of solvent by using a circulation pump. The extraction mass temperature was cooled to 25-30° C. and filtered through a 5-micron SS candle filter and the filtrate was collected in a cleaned receiver. The above extraction step was repeated two more times with 4-5 KL of the same solvent. The combined filtrate was concentrated in a clean reactor at 65-70° C. under vacuum until the extract was free of solvent residue. Weight of the extract was about 50-65 kg.

The extract obtained from above process was dissolved in 500-600 L of demineralized water in a 1.0 KL clean reactor under stirring. The temperature was slowly raised up to 55-60° C. 10% w/v NaOH solution was added the reaction mass pH raised to 11.5-12.0. The reaction temperature was maintained at 55-60° C. for about 1-1.5 h. The reaction mass was cooled to 25-30° C. and 5% v/v HCl solution was added to adjust the pH to 6.0-6.5. Stirring was continued about 1-1.5 h and the solution was kept for settling about 24-36 h at room temperature. The bottom-settled layer was collected and washed with 10-15 L of demineralized water 2-3 times and suck-dried under vacuum. The resulting wet cake was further dried in a vacuum tray drier at 65-70° C. until dry. The weight of extract was about 1.25±0.25 kg. The extract obtained was used in the following examples.

### EXAMPLE 2

### Quantification of β-Sitosterol in the Extract

### Instrumentation and Chromatographic Conditions

HPLC was performed on a Shimadzu LC2030 C Prominence-i (Japan) system equipped with a quaternary low-pressure gradient solvent delivery LC2030 pump with high-pressure switching valves, online LC2030 degasser unit, a high sensitivity LC2030 ultraviolet (UV) detector, high speed drive LC2030 autosampler with a 100 μL loop, and accommodated 216 samples at a time with a direct access rack system and large capacity column oven. The system was controlled and data analyzed by LabSolutions software. A separation was carried out in a Kinetex XBC-18 column (100 A°, 250 mm×4.6 mm, 5 μm pore size). The mobile phase consisted of isocratic elution with a low-pressure gradient using acetonitrile: methanol (05:95) with a flow rate of 1.5 mL/min and an injection volume of 10 μL. All solutions were degassed and filtered through a 0.45 μm pore size filter. The column was maintained at 30° C. throughout analysis, and the UV detector was set at 204 nm. 100% methanol was used as a diluent for assay by HPLC and the total liquid chromatography (LC) run time was 15 min. Using these chromatographic conditions, it was possible to confirm the retention time (RT) of β-sitosterol by injection of a corresponding standard separately.

### Preparation of Standard Solution

Accurately weighed 10.0 mg of β-sitosterol reference standard (Sigma Aldrich USA) was taken in a 25 ml standard volumetric flask and dissolved in methanol to obtain a stock concentration of 400 ppm. The standard solution was filtered through a 0.2 μ nylon syringe filter for injection.

### Preparation of Sample Solution

Weighed accurately around 15.0-20.0 mg of sample into a 25 mL volumetric flask, dissolved by adding methanol, sonicated and made up to the volume with methanol to get a final concentration in the range of 600-800 ppm. The sample solution was filtered through 0.2 μ nylon syringe filter for injection.

### Calculation

$$Assay \% = \frac{Peak area of the sample \times Conc . of the STD \times purity of the STD}{Peak area of the standard \times Conc . of the sample}$$

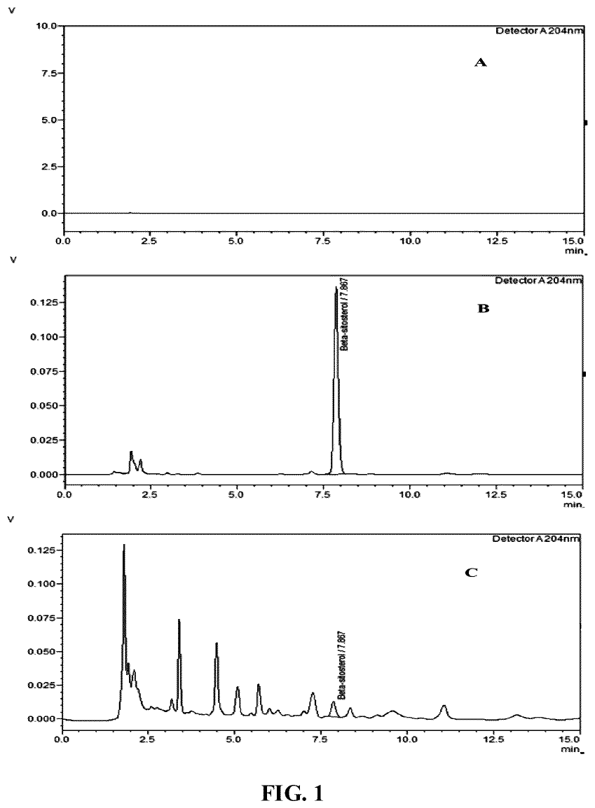

HPLC analysis revealed that the retention time of β-sitosterol standard and sample peak were found to be at 7.807 and 7.867, respectively (FIG. 1). The content of (3-sitosterol was 3.5±0.5 w/w% by HPLC method.

### Fingerprint Analysis of Phytosterols in A. heterophyllus by LCMS/MS Method

### Instrumentation and Chromatographic Conditions

The fingerprint analysis of phytosterols was operated using triple quadrupole mass spectrometry (LC-MS/MS-8050, Shimadzu, Japan) equipped with atmospheric pressure chemical ionization (APCI) source operating in positive ionization mode. The separation was carried out in a Kinetex Cis column (100 Å, 2.6 μm, 150×2.1 mm,). The mobile phase consists of isocratic elution using methanol: acetonitrile (1:99) with a flow rate of 0.3 mL/min and the injection volume of 5 μL. All solutions were degassed and filtered through 0.2 μm pore size filter. The column was maintained at 40° C. throughout the analysis. 100% methanol was used as a diluent and the total run time was 10 min. The MS chromatographic conditions used in the study were as follows: nebulizer gas flow, 3 L/min and drying gas flow, 5 L/min. The interface voltage and CID gas pressure were set at 4.5 Kv and 270 kPa. The interface temperature was maintained at 350° C., while the desolvation line (DL) and heat block (interface) temperature were maintained at 200° C. in the APCI source.

The ion transitions of the precursor to the product ion and their collision induced dissociated (CID) energy were as follows: the protonated ions [M+H]⁺ at m/z 379.10→69.15 ion as a quantifier (collision energy, −30 eV; dwell time, 30 ms; Q1 Pre-Bias, −19V; Q3 Pre-Bias, −14V), 379.10→338.30 ion as a qualifier-1 and 379.10→83.10 ion as a qualifier-2 for Ergosterol.

The protonated ions [M+H]⁺ at m/z 427.30→109.10 ion as a quantifier (collision energy, −27 eV; dwell time, 30 ms; Q1 Pre-Bias, −12V; Q3 Pre-Bias, −21V), 427.30→95.0 ion as a qualifier-1 and 427.30→121.05 ion as a qualifier-2 for Lupeol.

The protonated ions [M+H]⁺ at m/z 403.30→137.10 ion as a quantifier (collision energy, −25 eV; dwell time, 30 ms; Q1 Pre-Bias, −19V; Q3 Pre-Bias, −16V), 403.30-475.25 ion as a qualifier-1 and 403.30→159.15 ion as a qualifier-2 for Campestenol.

The protonated ions [M+H]⁺ at m/z 383.30→95.20 ion as a quantifier (collision energy, −34 eV; dwell time, 30 ms; Q1 Pre-Bias, −12V; Q3 Pre-Bias, −18V), 383.30→161.10 ion as a qualifier-1 and 383.30→81.20 ion as a qualifier-2 for Campesterol.

The protonated ions [M+H]⁺ at m/z 395.40→54.95 ion as a quantifier (collision energy, −53 eV; dwell time, 30 ms; Q1 Pre-Bias, −13V; Q3 Pre-Bias, −10V), 395.40→69.05 ion as a qualifier-1 and 395.40→132.95 ion as a qualifier-2 for Stigmasterol.

### Preparation of Sample Solution

25 mg of dried sample was weighed into 25 ml of a volumetric flask and dissolved by adding methanol, sonicated and made up to the volume with diluent to get a final concentration in the range of 1000 ppm. The sample solution was filtered solution through a 0.2 micron nylon syringe filter for injection.

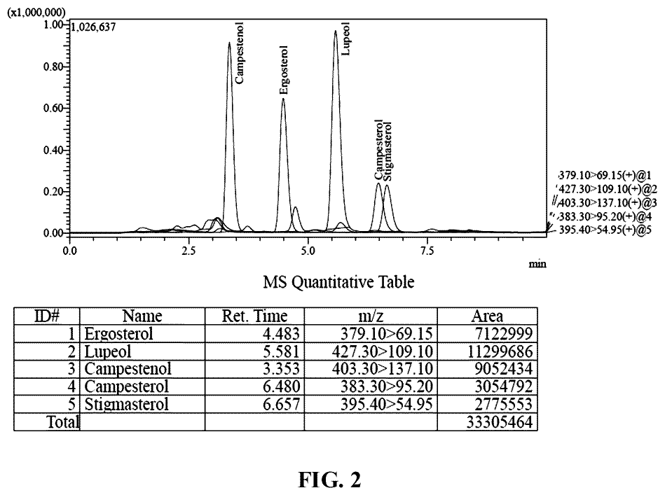

Qualitative analysis for flavonoids revealed the presence of ergosterol, lupeol, campestenol, campesterol, and stigmasterol in the extract (FIG. 2).

### Fingerprint Analysis of Flavonoids in in the Extract by LCMS/MS Method

### Instrumentation and Chromatographic Conditions

The fingerprint analysis of flavonoids was operated using triple quadrupole mass spectrometry (LC-MS/MS-8050, Shimadzu, Japan) equipped with atmospheric pressure chemical ionization (APCI) source operating in positive ionization mode. The separation was carried out in a Kinetex C18 column (100 Å, 2.6 μm, 150×2.1 mm,) and a gradient elution with a flow rate of 0.5 mL/min. The mobile phase consisted of two different solutions: 0.2% formic acid and acetonitrile which acted as solution A and solution B. All solutions were degassed and filtered through a 0.2 μm pore size filter. The gradient elution initial conditions were 30% of eluent B with linear gradient to 70% from 0.01 to 2 min, followed by linear gradient to 100% of eluent B at 4 min and this proportion was maintained for 2 min. The mobile phase composition returned to the initial condition at 8 min and was allowed to run for another 2 min before the injection of another sample.

All solutions were degassed and filtered through 0.2 μm pore size filter. The column was maintained at 40° C. throughout the analysis. 100% methanol was used as a diluent and the total run time was 10 min. The MS chromatographic conditions used in the study were as follows: nebulizer gas flow, 3 L/min and drying gas flow, 5 L/min. The interface voltage and CID gas pressure were set at 4.5 Kv and 230 kPa. The interface temperature was maintained at 350° C., while the desolvation line (DL) and heat block (interface) temperature were maintained at 200° C. in the APCI source.

The ion transitions of the precursor to the product ion and their collision induced dissociated (CID) energy were as follows: the protonated ions [M−H]⁺ at m/z 301.15→255.25 ion as a quantifier (collision energy, +18 eV; dwell time, 100 ms; Q1 Pre-Bias, +15V; Q3 Pre-Bias, +13V), 301.15→45.05 ion as a qualifier-1 and 301.15→257.0 ion as a qualifier-2 for artocarpanone.

The ion transitions of the precursor to the product ion and their collision induced dissociated (CID) energy were as follows: the protonated ions [M−H]⁺ at m/z 435.20→389.10 ion as a quantifier (collision energy, +17 eV; dwell time, 100 ms; Q1 Pre-Bias, +21V; Q3 Pre-Bias, +15V), 435.20→417.15 ion as a qualifier-1 and 435.20→402.20 ion as a qualifier-2 for artocarpin.

The ion transitions of the precursor to the product ion and their collision induced dissociated (CID) energy were as follows: the protonated ions [M−H]⁺ at m/z 433.15→387.25 ion as a quantifier (collision energy, +16 eV; dwell time, 100 ms; Q1 Pre-Bias, +13V; Q3 Pre-Bias, +20V), 433.15→375.10 ion as a qualifier-1 and 433.15→148.95 ion as a qualifier-2 for cycloartocarpin.

In summary, the qualitative analysis for phytosterols revealed for the presence of artocarpanone, artocarpin and cycloartocarpin in the extract (FIG. 3).

### EXAMPLE 3

### In Vitro 5-Alpha-Reductase Assay

In vitro 5-alpha-reductase assay was performed according to Sun et al. (1998) with some modifications. NADPH has characteristic absorbance at 340 nm, and the absorbance spectrum may be used to measure the decline in NADPH concentration as a measure of enzyme activity. Briefly, the reaction mixture contained 40 mM phosphate buffer pH 7.0, 93 μM NADPH, 2.265 μM testosterone and 20 μg liver S9 fraction with or without different concentrations of the extract (2-6 μg/mL) or reference compound Finasteride (20-70 ng/mL), incubated for 6 minutes at 37° C. The absorbance was read at 340 nm in UV-visible spectrophotometer. IC50 values were determined for the extract and Finasteride.

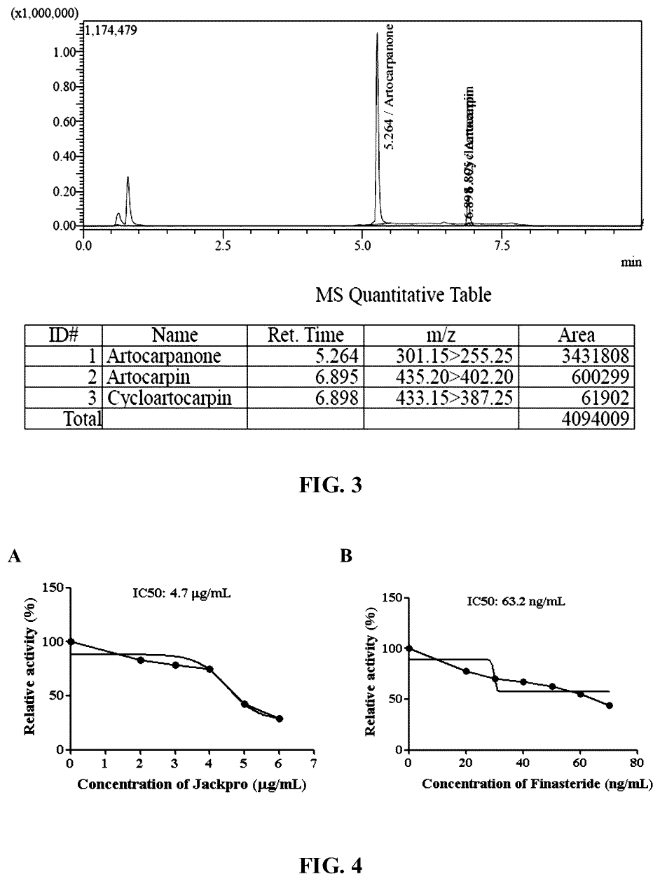

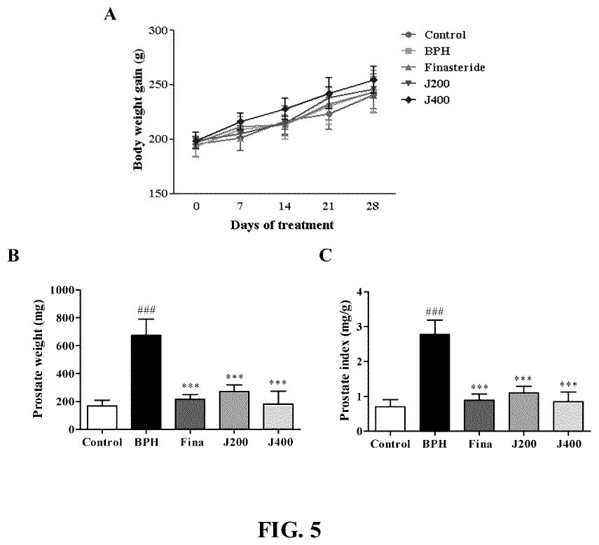

The extract was found to have profound inhibitory effect on 5-alpha-reductase activity (FIG. 4). The relative enzyme activity was decreased in a concentration dependent manner. The IC50 value for the extract was observed to be 4.7 μg/mL. Finasteride showed the IC50 value of 63.2 ng/mL.

### EXAMPLE 4

### Animals

Thirty male Wistar rats (8-9-week-old) were purchased from Biogen, Banglaore, India (CPCSEA Reg. No.971/bc/06). The animals were kept in polypropylene cages and housed in a controlled environment (temperature: 22±2° C. and relative humidity: 30-70%). Water and standard pellet diet were given ad libitum. After a 7-week acclimatization, the animals were randomized into five groups, each group consisting of six animals with less than 20% mean body weight range.

### BPH Induction and Dosage

All the animals except the normal control group were treated with testosterone 5 mg/kg subcutaneously to induce BPH for 28 days. Further, the rats in the treatment groups received a simultaneous oral treatment with reference drug Finasteride (1 mg/kg) or respective doses of the extract (200 and 400 mg/kg b.w.) (Table 1). At the end of the treatment, the animals were euthanised by overdose of gaseous anaesthesia (Isoflurane). Blood was collected by cardiac puncture. The vital parameters such as prostate weight, prostate index, serum testosterone level, histopathological examination of prostate tissues and antioxidant status were evaluated.

TABLE 1
Experimental design to evaluate the efficacy of the extract
in testosterone induced BPH model rats (n = 6).

| Group | Treatment (28 days) | Dose levels & route |
|---|---|---|
| Normal | Physiological saline (Vehicle) | 10 ml/kg & oral route |
| control | | |
| BPH control | Testosterone | 5 mg/kg & subcutaneous |
| Fina | Finasteride | 1 mg/kg |
| J200 | A. heterophyllus extract | 200 mg/kg & oral route |
| J400 | A. heterophyllus extract | 400 mg/kg & oral route |

### Prostate Growth in BPH Model Rats

The body weight gain of animals throughout the experimental period was recorded. There was no significant change in body weight observed among the rats in different treatment groups (FIG. 5A). At the end of the 28-day treatment, prostate tissues were excised, rinsed and weighed immediately after removal. The prostate weights were recorded, and the prostate index was calculated.

The mean prostate weight of testosterone induced BPH group were significantly increased as compared to normal rats (p<0.001). The prostate weights were reduced significantly in Finasteride and the extract treatment groups in comparison with BPH control (p<0.001) (FIG. 5B). A similar trend was observed in the prostate index. The extract (200 and 400 mg/kg doses) showed a dose-dependent reduction in prostate index compared to the BPH group (p<0.001) (FIG. 5C). A growth reduction of 90.22% was observed in the Finasteride group. In the extract-treated animals, the inhibition rate was 77.7% and 94.53% respectively at low and high dose groups (Table 2).

TABLE 2
Effect of extract administration on
prostate growth in BPH model rats

| Group | Growth inhibition (%) |
|---|---|
| Normal control | — |
| BPH control | — |
| Fina | 90.22 ± 10.85 |
| J200 | 77.70 ± 16.13 |
| J400 | 94.53 ± 22.75 |

Data are mean ± Standard deviation (n = 6)

Blood samples collected at the end of experiment were allowed to stand at room temperature for 20 min. Serum was separated by centrifuging at 3000 g, 4° C. All the samples were extracted by protein precipitation method. Briefly, 50 μL of serum samples were mixed with 200 μL of HPLC grade methanol. The samples were then centrifuged at 5000× g to precipitate proteins. A volume of 100 μL of supernatant was transferred into a sample vial and 10 μL was injected into the LC-MS/MS system.

Testosterone was quantified using triple quadrupole mass spectrometry (LC-MS/MS-8050, Shimadzu, Japan) equipped with electrospray ionization (ESI) source operating in positive ionization mode. Nitrogen was used as nebulizer, drying and heating gas. Argon gas was used for the collision induced dissociation (CID) gas. The separation was carried out in a Kinetex C18 column (100 Å, 2.6 μm, 150×2.1 mm,) at a flow rate of 0.2 mL/min in ESI source. The mobile phase consisted of 0.2% formic acid: acetonitrile (A: B)=30: 70 with isocratic elution and an injection volume of 5 μL. All solutions were degassed and filtered through 0.2 μm pore size filter. The column was maintained at 40° C. throughout the analysis. 100% methanol was used as a diluent and the total run time was 5 min. The MS chromatographic conditions used in the study were as follows: nebulizer gas flow, 3 L/min; drying gas flow, 10 L/min and heating gas flow, 10 L/min. The interface voltage and CID gas pressure were set at 4 Kv and 270 kPa. The interface temperature was maintained at 300° C., while the desolvation line (DL) and heat block (interface) temperature were maintained at 250 and 400° C. respectively in the ESI source. Other parameters were tuned automatically.

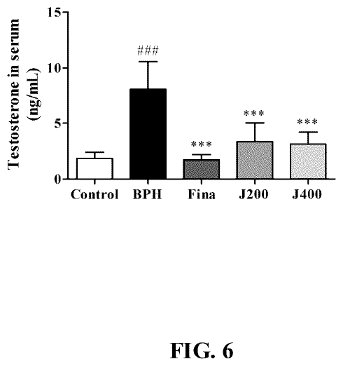

The serum testosterone level was significantly higher in the BPH group (8.03±2.53 ng/mL) compared to the control group rats (1.86±0.55 ng/mL) (p<0.001). The level of testosterone was markedly decreased in the treatment groups as compared to the BPH group (p<0.001). The serum testosterone in the extract-treated groups were 3.36±1.67 ng/mL and 3.13±1.07 ng/mL respectively (FIG. 6).

### 5-Alpha-Reductase Activity in Prostate

The 5-alpha-reductase activity was determined in prostate tissue samples from the experimental groups. Briefly, the reaction mixture contained 40 mM phosphate buffer pH 7.0, 93 μM NADPH, 2.265 μM testosterone and 100 μg prostate tissue homogenate, incubated for 6 minutes at 37° C. NADPH has a characteristic absorbance at 340 nm, and the absorbance spectrum may be used to identify NADPH as a kind of the substrate in this enzymatic reaction by measuring the declining substrate concentration. The absorbance was read at 340 nm. The enzyme activity was expressed as pmol/min/mg protein.

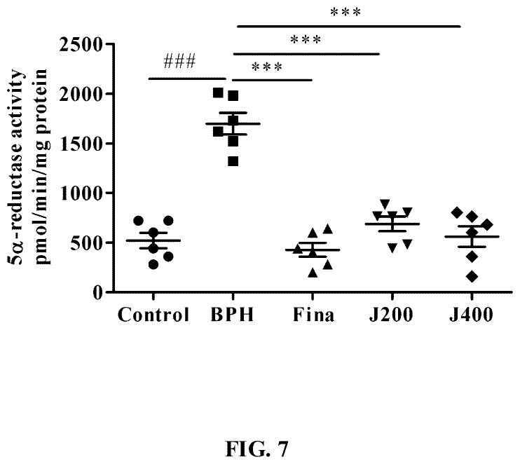

5-alpha-reductase activity was found have increased significantly in the BPH group compared to normal rats (p<0.001). Interestingly, the extract treatment significantly inhibited the enzyme activity compared to untreated BPH group (p<0.001). The results were comparable to the reference drug Finasteride (FIG. 7).

### Histopathological Examination of Prostate Tissue

The paraffin embedded prostate tissue samples were cut into 5 μm thickness. The sections were stained with hematoxylin and eosin (H&E, Sigma-Aldrich, St. Louis, MO, USA). The images were captured using a microscope (Leica, Germany).

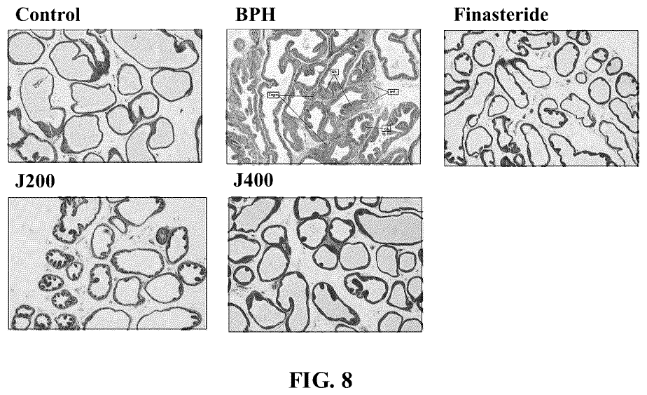

Prostate tissues of control rats showed normal glandular architecture while in the untreated BPH group, a decreased luminal area was seen indicating glandular hyperplasia. Further, the hyperplastic patterns such as nuclear stratification, thicker epithelium, and glandular necrosis with inflammation were evident in the BPH group. These pathological changes were not evident in the Finasteride or extract-treated groups. In the extract-treated groups, the epithelial cell thickness was reduced, and the luminal area increased considerably (FIG. 8).

### Antioxidant Activity in Prostate Tissue

Oxidative stress often leads to compensatory cellular proliferation resulting in BPH. The antioxidant status in the prostate tissue homogenates of the BPH model rats was investigated.

For the total superoxide dismutase (SOD) assay, a 3.0 mL reaction mixture contained 50 mM sodium carbonate (pH 10.2), 96 μNBT, 0.1 mM EDTA, 20 mM hydroxylamine hydrochloride, 0.03% (v/v) Triton X-100 and 50 μL prostate tissue homogenate. The absorbance was recorded at 560 nm for 5 minutes. One unit of SOD activity was defined as the amount of enzyme required that caused 50% of NBT reduction at 25° C.

Catalase activity was determined following the method of Aebi (1984).

GSH levels were determined by the method of Ellman (1959) with slight modification. Briefly, 100 μL of homogenate was mixed with 100 μL of 10 mM Ellman's reagent. The volume was made up to 1 mL using 0.1 M phosphate buffer with 5 mM EDTA, pH 7.4 and the yellow color that developed was read at 412 nm. The concentration of GSH in the tissue homogenate was determined by calculating from the linear equation or the regression curve generated from GSH standard.

Glutathione reductase catalyses the reduction of glutathione disulfide (GSSG) to the sulfhydryl form glutathione (GSH), which is a critical molecule in resisting oxidative stress and maintaining the oxidative balance of the cell. The activity of glutathione reductase in the prostate homogenate was measured by the method described by Dringen and Gutterer (2002). The enzyme activity was determined by the time dependent change in NADPH (ε=6270 M⁻¹ cm⁻¹) at 340 nm after the addition of the sample.

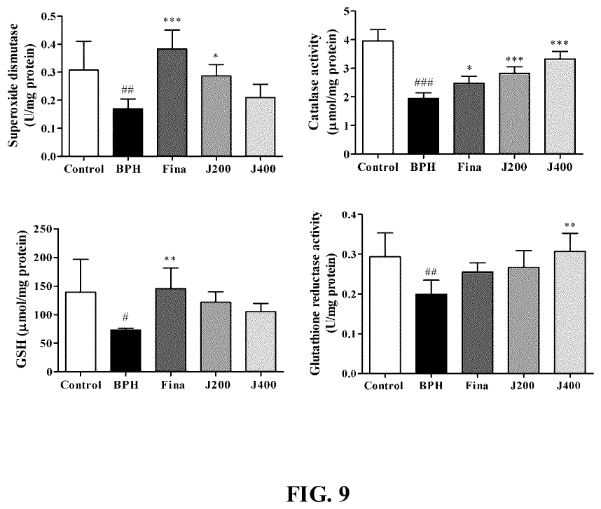

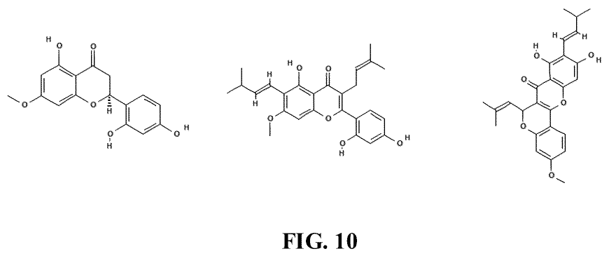

As expected, the activity of antioxidant enzymes was reduced significantly in the untreated BPH group compared to the normal control group (p<0.05). Treatment with Finasteride (p<0.001) and the extract at 200 mg/kg (p<0.05) significantly increased the SOD activity in comparison with BPH group. A similar trend was observed in GSH levels. Catalase and glutathione reductase activities increased dose-dependently in the extract-treated BPH groups. The data were significant in the extract treatment group at 400 mg/kg when compared to the untreated BPH rats (p<0.01) (FIG. 9).

In summary, the extract was effective in ameliorating conditions in testosterone-induced BPH model rats as a function of 5-alpha-reductase inhibition.

### EXAMPLE 5

### Molecular Docking Analysis

The pharmacologically active flavonoids artocapanone, artocarpin and cycloartocarpin present in the extract were used in this study. The 3D structures of compounds were obtained from Pubchem (https://pubchem.ncbi.nlm.nih.gov). The drug-likeliness of the molecules was determined using SWISSADME prediction (http://www.swissadme.ch/). 3D coordinates were prepared using PRODRG server.

In silico docking analysis was performed using Autodock 4.2 tools. As the crystal structure of 5-alpha-reductase is not available, was modelled 5β-reductase due to similar substrate specificity. Also, both the proteins have similar enzymatic function in metabolising steroid hormones (Yao et al. 2011). Molecular docking was used to predict the interactive pattern of flavonoids present in the extract with the enzyme active site.

The crystal structure of human liver 5β-reductase (PDB ID: 3G1R) was downloaded in .pdb format from PDB database (http://www.rcsb.org/). Chain B of the protein was used for macromolecule preparation. The coordinates of PDB structures were prepared for molecular docking by removing the water ions and ligands using the Python molecule viewer.

AutoDock tools were utilized to generate grids, calculate dock score, and evaluate the conformers of compounds bound in the active site of the enzyme. The grid map was centred at active site residues of the protein and generated with AutoGrid. The Lamarckian genetic algorithm and the pseudo-Solis and Wets methods were applied for minimization, using default parameters.

### Results

The drug-like nature of the compounds was confirmed as the molecules satisfied the Lipinski's rule of five (molecular weight, polar surface area, lipophilicity, hydrogen bonding and charge) (Table 3).

TABLE 3
Drug likeliness of vital flavonoids of the extract

| | Compounds | | |
|---|---|---|---|
| Lipinski's rule of five | Artocarpanone | Artocarpin | Cycloartocarpin |
| Molecular weight | 302.28 | 436.5 | 434.48 |
| (<500 Da) | | | |
| MLog P (<4.15) | 0.41 | 2.3 | 2.3 |
| H-Bond donor (<5) | 3 | 3 | 2 |
| H-Bond acceptor (<10) | 6 | 6 | 6 |
| Violation | 0 | 0 | 0 |

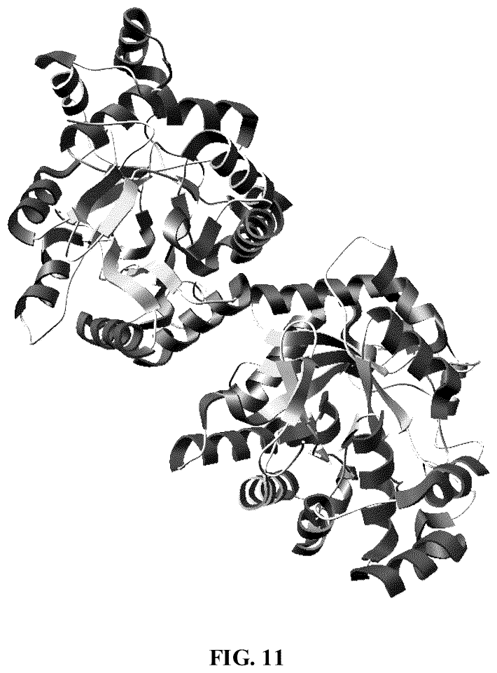

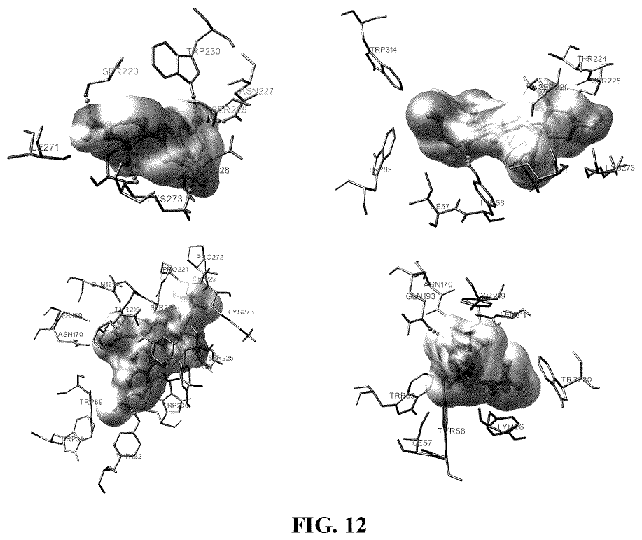

Further, the molecular docking analysis revealed profound interaction between the tested compounds and 5β-reductase active site amino acid residues (FIG. 10). Among the three compounds, cycloartocarpin demonstrated stronger interaction with the enzyme active site, having a binding energy of −9.02 kcal/mol (Ki=250 nM). The inhibitory constant was comparable to reference compound finasteride (Ki=120 nM) (Table 4).

TABLE 4
Molecular docking analysis of bioactive compounds of the extract
and Finasteride into the active site of 5β-reductase

| | | | Inhibition | | VDW-H Bond |
|---|---|---|---|---|---|
| | Binding | Ligand | constant | Intermolecular | Desolvation |
| Compound | energy | efficiency | (μM) | energy | energy |
| Artocarpanone | −6.99 | −0.32 | 7.55 | −8.48 | −8.2 |
| Artocarpin | −7.28 | −0.23 | 4.62 | −10.56 | −10.5 |
| Cycloartocarpin | −9.02 | −0.28 | 0.25 | −11.4 | −11.24 |
| Finasteride | −9.45 | −0.35 | 0.12 | −10.05 | −9.97 |

### REFERENCES

1. Bidlack W R, Omaye S T, Meskin M S, Topham D K W. Phytochemicals as bioactive agents. CRC Press, UK, 2016

2. Cicero A F G, Allkanjari O, Busetto GM, Cai T, Larganà G, Magri V, et al. Nutraceutical treatment and prevention of benign prostatic hyperplasia and prostate cancer. Arch Ital Urol Androl. 2019; 91(3)

3. Govema P, Giachetti D, Biagi M, Manetti F, De Vico L. Hypothesis on Serenoa repens (Bartram) small extract inhibition of prostatic 5a-reductase through an in silico approach on 5α-reductase x-ray structure. Peer J. 2016; 4: e2698

4. Jena A K, Vasisht K, Sharma N, Kaur R, Dhingra MS , Karan M. Amelioration of testosterone induced benign prostatic hyperplasia by Prunus species. J Ethnopharmacol. 2016; 190: 33-45

5. Mohammadi A, Mansoori B, Aghapour M, Baradaran B. Urtica dioica dichloromethane extract induce apoptosis from intrinsic pathway on human prostate cancer cells (PC3). Cell Mol Biol (Noisy-le-grand). 2016; 62(3): 78-83

6. Jagadeesh S L, Reddy B S, Swamy G S K, Gorbal K, Hegde L, Raghavan G S V. Chemical composition of jackfruit (Artocarpus heterophyllus Lam.) selections of Western Ghats of India. Food Chem. 2007; 102(1): 361-365

7. Chandrika U G, Jansz E R, Warnasuriya N D. Analysis of carotenoids in ripe jackfruit (Artocarpus heterophyllus) kernel and study of their bioconversion in rats. J Sci Food Agric. 2004; 85(2): 186-190

8. Venkataraman K. Wood of phenolics in the chemotaxonomy of the Moraceae. Phytochem. 2001; 11(5): 1571-1586

9. Wong K C, Lim C L, Wong L L. Volatile flavour constituents of chempedak (Artocarpus polyphema Pers.) fruit and jackfruit (Artocarpus heterophyllus Lam.) from Malaysia. Flavour Frag J. 1992; 7(6): 307-311

10. Jagtap U B, Panaskar S N, Bapat V A. Evaluation of antioxidant capacity and phenol content in jackfruit (Artocarpus heterophyllus Lam.) fruit pulp. Plant Foods Hum Nutr. 2010; 65(2): 99-104

11. Shanmugapriya K, Saravana P S, Payal H, Peer Mohammed S, Binnie W. Antioxidant activity, total phenolic and flavonoid contents of Artocarpus heterophyllus and Manilkara zapota seeds and its reduction potential. Int J Pharm Pharmaceutical Sci. 2011; 3(5): 256-260.

12. Roehrborn C G. Male lower urinary tract symptoms (LUTS) and benign prostatic hyperplasia (BPH). Med Clin North Am 2011; 95: 87-100

13. Wu C, Kapoor A. Dutasteride for the treatment of benign prostatic hyperplasia. Expert Opin on Pharmacother 2013; 14: 1399-1408

14. Schauer I G, Rowley D R. The functional role of reactive stroma in benign prostatic hyperplasia. Differentiation 2011; 8: 200-210

15. Chughtai B, Forde J C, Thomas D D M, Laor L, Hossack T, Woo H H, et al. Benign prostatic hyperplasia. Nat Rev Dis Primers 2016; 16031

16. Sun Z Y, Zheng W J, Feng J, Tu Z H. A convenient and rapid method to study enzymatic kinetics of steroid 5a-reductase inhibitors, Indian journal of Pharmacology 1998; 257-2262

17. Yao Z, Xu Y, Zhang M, Jiang S, Nicklaus M C, Liao C. Discovery of a novel hybrid from finasteride and epristeride as 5alpha-reductase inhibitor. Bioorg Med Chem Lett 2011; 21(1): 475-478

18. Baliga M S, Shivashankara A R, Haniadka R, Dsouza J, Bhat H P. Phytochemistry, nutritional and pharmacological properties of Artocarpus heterophyllus Lam. (Jackfruit): A review. Food Res Int. 2011; 44(9): 1800-1811

1. A method of promoting urinary tract health, comprising administering to a subject in need thereof an effective amount of a composition comprising an extract of Artocarpus heterophyllus.

2. The method of claim 1, wherein said extract comprises Artocarpus heterophyllus leaf extract.

3. The method of claim 1, wherein said extract comprises at least one of artocarpanone, artocarpin, or cycloartocarpin.

4. The method of claim 1, wherein said extract comprises at least one of ergosterol, lupeol, campestenol, campesterol, and stigmasterol.

5. The method of claim 1, wherein said subject has a prostate disorder.

6. The method of claim 5, wherein said prostate disorder is benign prostatic hyperplasia, prostatitis, or a combination thereof.

7. The method of claim 1, wherein said subject has one or more of an enlarged prostate, increased prostate weight, prostate inflammation, inhibited apoptosis of prostate tissue, and prostate growth.

8. The method of claim 1, wherein said subject has one or more lower urinary tract symptoms selected from urgent need to urinate, increased frequency of urination, difficulty starting urination, weak urine stream, urine stream that stops and starts, dribbling at the end of urination, involuntary urination, painful urination, inability to completely empty the bladder, urinary tract infection, inability to urinate, and blood in the urine.

9. The method of claim 1, wherein administering said composition reduces serum testosterone in said subject.

10. The method of claim 9, wherein administering said composition reduces said serum testosterone by about 58% to about 61%.

11. The method of claim 1, wherein said composition is administered systemically.

12. The method of claim 1, wherein said composition is administered orally.

13. The method of claim 1, wherein said subject is administered between about 200 mg/kg body weight and about 400 mg/kg body weight of said extract.

14. The method of claim 1, wherein said composition is combined with a nutritional supplement, food supplement, or beverage.

15. The method of claim 1, wherein said composition comprises an excipient.

16. The method of claim 1, wherein said excipient is artificial.

17. The method of claim 1, wherein said subject is a human.

18. The method of claim 1, wherein said subject is male.
